(19) United States
(12) Patent Application Publication
(10) Pub. No.: US 2011/0251134 A1
(43) Pub. Date: Oct. 13, 2011

(54) COMBINATION OF (A) A DNA TOPOSOMERASE INHIBITOR AND (B) AN IAP INHIBITOR

(76) Inventor: Leigh Zawel, Hingham, MA (US)

(21) Appl. No.: 10/587,758

(22) PCT Filed: Feb. 4, 2005
(86) PCT No.: PCT/EP05/01180

§ 371 Date: May 30, 2007

Related U.S. Application Data
(60) Provisional application No. 60/541,984, filed on Feb. 5, 2004.

(51) Int. Cl. A61K 38/06; A61P 35/04; A61P 35/00

(52) U.S. Cl. 514/18.9

(57) ABSTRACT

The invention relates to a pharmaceutical combination which comprises (a) a DNA topoisomerase inhibitor compound and (b) a compound that inhibits the caspase-9 inhibiting properties of an inhibitor of apoptosis protein (IAP) for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The invention relates to a pharmaceutical combination which comprises (a) a DNA topoisomerase inhibitor compound and (b) a compound (IAP inhibitor) that inhibits the caspase-9 inhibiting properties of an inhibitor of apoptosis protein (IAP) and optionally at least one pharmaceutically acceptable carrier for simultaneous, separate or sequential use, in particular for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human. A greater than additive effect is seen when compounds (a) and (b) are used in combination.

DNA topoisomerases are enzymes essential for the relaxation of DNA during a number of critical processes, including replication, transcription, and repair. There are two types of topoisomerases; topoisomerase I and topoisomerase II. Camptothecin and related compounds are the most important inhibitors of topoisomerase I.

Camptothecin is a plant alkaloid of the following structure

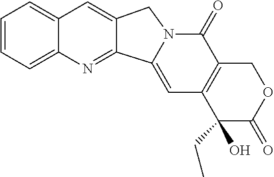

Irinotecan and topotecan are related compounds that are approved for treatment of certain cancers. In addition, several topoisomerase I inhibitors that are structurally related to camptothecin are in development, including BNP1350, SN38, 9-amino-camptothecin, lurtotecan, gimatecan, several homocamptothecins, such as diflomotecan, and several conjugates, usually via the 20S hydroxy or a 10 hydroxy, with, for example, carboxymethyldextran, poly-L-gutamic acid, polyethylene glycol and the like, such as T-0128, DX-310, CT-2106 and Protecan.

A recently reported molecular mechanism for circumvention of apoptosis involves the overexpression of members of the IAP family. IAPs sabotage apoptosis by directly interacting with and neutralizing Caspases. The prototype IAP, XIAP, has three functional domains referred to as BIR 1, 2 & 3 domains. BIR3 interacts directly with Caspase 9 and inhibits its ability to bind and cleave its natural substrate, Procaspase 3. Thus, in an important embodiment of this invention, IAP inhibitor compound inhibits the interaction between the BIR3 domain of XIAP and Caspase-9.

Therapeutic compounds that inhibit the interaction between the BIR3 domain of XIAP and Caspase-9 include mimetics of SMAC and antisense nucleic acids, for example as claimed in U.S. Pat. No. 6,300,492.

Mimetics of SMAC include compounds described in WO2004/005248, in particular compound C

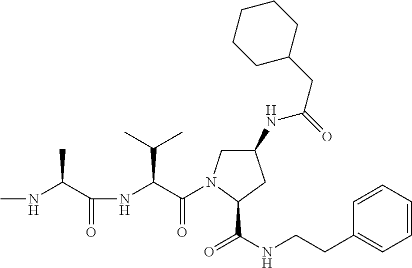

or compound D:

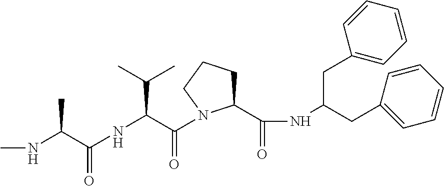

The term “topoisomerase II inhibitors” as used herein includes, but is not limited to the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide.

Hence, the present invention also pertains to a combination such as a combined preparation or a pharmaceutical composition which comprises (a) a DNA topoisomerase inhibitor and (b) an IAP inhibitor. More particularly, in a first embodiment, the present invention relates to a combination which comprises (a) a topoisomerase I inhibitor and (b) an IAP inhibitor, and in a second embodiment, the present invention relates to a combination which comprises (a) a topoisomerase II inhibitor and (b) an IAP inhibitor.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single.

In one embodiment of the invention, (a) the topoisomerase I inhibitor is selected from the group consisting of Camptothecin, Irinotecan and topotecan and related compounds that are approved for treatment of certain cancers, BNP1350, SN38, 9-amino-camptothecin, lurtotecan, gimatecan, several homocamptothecins, such as diflomotecan, and several conjugates, usually via the 20S hydroxy or a 10 hydroxy, with, for example, carboxymethyldextran, poly-L-glutamic acid, polyethylene glycol and the like, such as T-0128, DX-310, CT-2106 and Protecan.

In one embodiment of the invention, (b) the IAP inhibitor is selected from the group consisting of the therapeutic compounds that inhibit the interaction between the BIR3 domain of XIAP and Caspase-9 such anti-sense nucleic acids, for example as claimed in U.S. Pat. No. 6,300,492. and mimetics of SMAC, for example as described in WO2004/005248, in particular compound C and compound D.

The term “treating” or “treatment” as used herein comprises the a treatment effecting a delay of progression of a disease. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

The term “solid tumor” especially means breast cancer, ovarian cancer, cancer of the colon and generally the GI (gastro-intestinal) tract, cervix cancer, lung cancer, in particular small-cell lung cancer, and non-small-cell lung cancer, head and neck cancer, bladder cancer, cancer of the prostate or Kaposi's sarcoma. The present combination inhibits the growth of solid tumors, but also liquid tumors. Furthermore, depending on the tumor type and the particular combination used a decrease of the tumor volume can be obtained. The combinations disclosed herein are also suited to prevent the metastatic spread of tumors and the growth or development of micrometastases. The combinations disclosed herein are in particular suitable for the treatment of poor prognosis patients, especially such poor prognosis patients having non-small-cell lung cancer.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It will be understood that references to the combination partners (a) and (b) are meant to also include the pharmaceutically acceptable salts. If these combination partners (a) and (b) have, for example, at least one basic center, they can form acid addition salts. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization.

A combination which comprises (a) a DNA topoisomerase inhibitor and (b) an IAP inhibitor, in which the active ingredients are present in each case in free form or in the form of a pharmaceutically acceptable salt and optionally at least one pharmaceutically acceptable carrier, will be referred to hereinafter as a COMBINATION OF THE INVENTION.

The pharmacological activity of a COMBINATION OF THE INVENTION may, for example, be demonstrated in a clinical study or in a test procedure as essentially described hereinafter. Suitable clinical studies are, for example, open label non-randomized, dose escalation studies in patients with advanced solid tumors. Such studies prove in particular the synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, the combination partner (a) is administered with a fixed dose and the dose of the combination partner (b) is escalated until the Maximum Tolerated Dosage is reached.

Irinotecan can be administered, e.g., in the form as it is marketed, e.g. under the trademark CAMPTOSAR™. Topotecan can be administered, e.g., in the form as it is marketed, e.g. under the trademark HYCAMTIN™. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™. Doxorubicin can be administered, e.g., in the form as it is marketed, e.g. under the trademark ADRIBLASTIN™. Epirubicin can be administered, e.g., in the form as it is marketed, e.g. under the trademark FARMORUBICINT™. Idarubicin can be administered, e.g., in the form as it is marketed, e.g. under the trademark ZAVEDOST™. Mitoxantrone can be administered, e.g., in the form as it is marketed, e.g. under the trademark NOVANTRONT™.

It is one objective of this invention to provide a pharmaceutical composition comprising a quantity, which is therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils, alcohols, flavoring agents, preservatives, coloring agents; or carriers such as starches, sugars, microcristalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The COMBINATION OF THE INVENTION can be a combined preparation or a pharmaceutical composition.

Moreover, the present invention relates to a method of treating a Warm-blooded animal having a proliferative disease comprising administering to the animal a COMBINATION OF THE INVENTION in a quantity which is therapeutically effective against said proliferative disease.

Furthermore, the present invention pertains to the use of a COMBINATION OF THE INVENTION for the treatment of a proliferative disease and for the preparation of a medicament for the treatment of a proliferative disease.

Moreover, the present invention provides a commercial package comprising as active ingredients COMBINATION OF THE INVENTION, together with instructions for simultaneous, separate or sequential use thereof in the delay of progression or treatment of a proliferative disease.

Preferred embodiments of the invention are represented by combinations comprising
compound C and topotecan,
camptothecin and compound D, or
compound C and camptothecin.

In further aspects, the present inventions provides
a combination which comprises (a) a COMBINATION OF THE INVENTION, wherein the active ingredients are present in each case in free form or in the form of a pharmaceutically acceptable salt or any hydrate thereof, and optionally at least one pharmaceutically acceptable carrier; for simultaneous, separate or sequential use;
a pharmaceutical composition comprising a quantity which is jointly therapeutically effective against a proliferative disease of a COMBINATION OF THE INVENTION and at least one pharmaceutically acceptable carrier;
the use of a COMBINATION OF THE INVENTION for the treatment of a proliferative disease;
the use of a COMBINATION OF THE INVENTION for the preparation of a medicament for the treatment of a proliferative disease;
the use of a combination COMBINATION OF THE INVENTION wherein the IAP inhibitor is selected from compound C and compound D;
the use of a COMBINATION OF THE INVENTION wherein the DNA topoisomerase inhibitor is a topoisomease I inhibitor; and
the use of COMBINATION OF THE INVENTION wherein the DNA topoisomerase inhibitor is a topoisomease II inhibitor.

In particular, the present invention relates to a combination comprising (a) a topoisomerase I inhibitor and (b) an IAP inhibitor.

Moreover, in particular, the present invention relates to a combined preparation, which comprises (a) one or more unit dosage forms of topoisomerase I inhibitor and (b) one or more unit dosage forms of an IAP inhibitor.

Furthermore, in particular, the present invention pertains to the use of a combination comprising (a) a topoisomerase I inhibitor and (b) an IAP inhibitor for the preparation of a medicament for the treatment of a proliferative disease.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

Irinotecan may be administered to a human in a dosage range varying from about 50 to 350 mg/m² day.

Topotecan may be administered to a human in a dosage range varying from about 1 to 5 mg/m² day.

Etoposide phosphate may be administered to a human in a dosage range varying from about 25 to 115 mg/m² day, e.g. 56.8 or 113.6 mg/m² day.

Teniposide may be administered to a human in a dosage range varying from about 75 to 150 mg about every two weeks.

Doxorubicin may be administered to a human in a dosage range varying from about 10 to 100 mg/m² day, e.g. 25 or 50 mg/m² day.

Epirubicin may be administered to a human in a dosage range varying from about 10 to 200 mg/m² day.

Idarubicin may be administered to a human in a dosage range varying from about 0.5 to 50 mg/m² day.

Mitoxantrone may be administered to a human in a dosage range varying from about 2.5 to 25 mg/m² day.

The following Examples illustrate the invention described above; they are not, however, intended to limit the scope of the invention in any way. The beneficial effects of the COMBINATION OF THE INVENTION can also be determined by other test models known as such to the person skilled in the pertinent art.

### EXAMPLE 1

In a melanoma model, compound D (250 nM) shows growth at about 90% of control, camptothecin (5 nM) shows growth of about 50% of control while the combination of compound D (250 nM) and camptothecin (5 nM) shows growth of less than 3% of control.

### EXAMPLE 2

In a breast tumor model, both compound C and topotecan (1 nM) individually increase caspase-3 activity less than two fold over the control. A nearly twelve fold increase in caspase-3 activity is seen with the same amount of compound C at a concentration of about 1 nM topotecan.

### EXAMPLE 3

In a metastatic melanoma cell line A2058, the following combination index (CI) values are obtained from synergy experiments conducted with camptothecin and compound C, camptothecin and compound D.

CI values for each compounds are calculated at ED90, ED75 and ED 50 for each combination partner.

| | Cl value |
|---|---|
| Very strong synergism | <0.1 |
| Strong synergism | 0.1-0.3 |
| synergism | 0.3-0.7 |
| Moderate synergism | 0.7-0.85 |
| Slight synergism | 0.85-0.9 |
| Nearly additive | 0.9-1.1 |
| antagonism | 1.1->10 |

| | Camptothecin | |
|---|---|---|
| | Compound D | Compound C |
| ED90 | 0.379 | 0.025* |
| ED75 | 0.249* | 0.034* |
| ED50 | 0.255* | 0.131* |

Values with a star (*) indicate strong synergism. The results show strong synergism between Compound D and Camptothecin and Compound C and Camptothecin.

1. A combination which comprises (a) a DNA topoisomerase inhibitor and (b) an IAP inhibitor, wherein the active ingredients are present in each case in free form or in the form of a pharmaceutically acceptable salt or any hydrate thereof, and optionally at least one pharmaceutically acceptable carrier; for simultaneous, separate or sequential use.

2. A pharmaceutical composition comprising a quantity which is jointly therapeutically effective against a proliferative disease of a combination according to claim 1 and at least one pharmaceutically acceptable carrier.

3. A method for the treatment of a proliferative disease comprising administering a combination as defined in claim 1.

4. (canceled)

5. A method according to claim 3 wherein the IAP inhibitor is selected from compound C 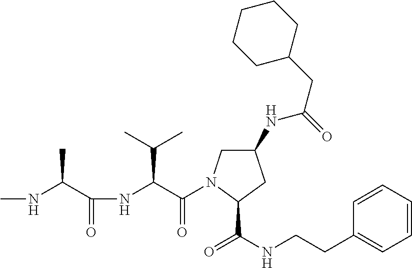
and compound D

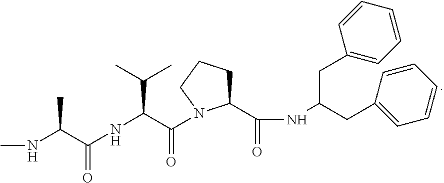

6. A method according to claim 3 wherein the DNA topoisomerase inhibitor is a topoisomease I inhibitor.

7. A method according to claim 3 wherein the DNA topoisomerase inhibitor is a topoisomease II inhibitor.

8. A method of treating a proliferative disease in a patient, which comprises administering to the patient an effective combination of (a) a DNA topoisomerase inhibitor and (b) an IAP inhibitor.

9. The method according to claim 8, which comprises administering to the patient an effective combination of (a) a topoisomease I inhibitor and (b) an IAP inhibitor.

10. A combined preparation, which comprises (a) one or more unit dosage forms of topoisomerase I inhibitor and (b) one or more unit dosage forms of an IAP inhibitor.

11. The method according to claim 8, which comprises administering to the patient an effective combination of (a) a topoisomease II inhibitor and (b) an IAP inhibitor.

12. A combined preparation, which comprises (a) one or more unit dosage forms of topoisomerase II inhibitor and (b) one or more unit dosage forms of an IAP inhibitor.
